(19) United States
(12) Patent Application Publication
(10) Pub. No.: US 2022/0287875 A1
(43) Pub. Date: Sep. 15, 2022

(54) Twist and Lock Connector for Gel Pads

(71) Applicant: C. R. Bard, Inc., Franklin Lakes, NJ (US)

(72) Inventors: Ana Minchew, Covington, GA (US); Jill Walthall Jones, Avondale Estates, GA (US); Gina Whitlock, Barrington Hills, IL (US); Cassie L. Singleton, Social Circle, GA (US); Sean E. Walker, Platteville, CO (US)

(21) Appl. No.: 17/691,990

(22) Filed: Mar. 10, 2022

Related U.S. Application Data
(60) Provisional application No. 63/159,852, filed on Mar. 11, 2021.

(51) Int. Cl. A61F 7/00; A61F 7/02; A61B 90/90

(52) U.S. Cl. CPC A61F 7/0085; A61F 7/02; A61B 90/90; A61F 2007/0054; A61F 2007/0219; A61F 2007/0095

(57) ABSTRACT

A targeted temperature management (TTM) system is disclosed that includes a TTM module configured to provide a TTM fluid and a thermal pad configured to facilitate thermal energy transfer between the TTM fluid and a patient. The system further includes a fluid delivery line (FDL) coupled with the TTM module at a proximal end. A hub at the distal end of the FDL includes a delivery hub connector coupled with the delivery conduit connector, a return hub connector couples with the return conduit connector, and a locking mechanism is selectively configurable to alternate between a release configuration and a lock configuration. When the locking mechanism is in the lock configuration, it prevents at least one of separation of the delivery conduit connector from the delivery hub connector or separation of the return conduit connector from the return hub connector.

### PRIORITY

This application claims the benefit of priority to U.S. Provisional Application No. 63/159,852, filed Mar. 11, 2021, which is incorporated by reference in its entirety into this application.

### BACKGROUND

The effect of temperature on the human body has been well documented and the use of targeted temperature management (TTM) systems for selectively cooling and/or heating bodily tissue is known. Elevated temperatures, or hyperthermia, may be harmful to the brain under normal conditions, and even more importantly, during periods of physical stress, such as illness or surgery. Conversely, lower body temperatures, or mild hypothermia, may offer some degree of neuroprotection. Moderate to severe hypothermia tends to be more detrimental to the body, particularly the cardiovascular system.

Targeted temperature management can be viewed in two different aspects. The first aspect of temperature management includes treating abnormal body temperatures, i.e., cooling the body under conditions of hyperthermia or warming the body under conditions of hypothermia. The second aspect of thermoregulation is an evolving treatment that employs techniques that physically control a patient's temperature to provide a physiological benefit, such as cooling a stroke patient to gain some degree of neuroprotection. By way of example, TTM systems may be utilized in early stroke therapy to reduce neurological damage incurred by stroke and head trauma patients. Additional applications include selective patient heating/cooling during surgical procedures such as cardiopulmonary bypass operations.

TTM systems circulate a fluid (e.g., water) through one or more thermal contact pads coupled with a patient to affect surface-to-surface thermal energy exchange with the patient. In general, TTM systems comprise a TTM fluid control module coupled with at least one contact pad via a fluid deliver line. One such TTM system is disclosed in U.S. Pat. No. 6,645,232, titled “Patient Temperature Control System with Fluid Pressure Maintenance” filed Oct. 11, 2001 and one such thermal contact pad and related system is disclosed in U.S. Pat. No. 6,197,045 titled “Cooling/heating Pad and System” filed Jan. 4, 1999, both of which are incorporated herein by reference in their entireties. As noted in the '045 patent, the ability to establish and maintain thermally intimate pad-to-patient contact is of importance to fully realizing medical efficacies with TTM systems.

A fluid delivery line generally includes at least two fluid conduits for transporting TTM fluid to and from the thermal pad. Fluid delivery lines may include connection systems for selectively connecting to and disconnecting from the thermal pad. The connections may be located in close proximity to the patient and thereby exposing the connection system to disturbance by the patient. Such disturbance may cause disconnection of the connection system and/or leakage of TTM fluid. Disclosed herein are systems, devices, and methods for securing the fluid connection between the thermal pad and the fluid delivery line.

### SUMMARY OF THE INVENTION

Briefly summarized, disclosed herein is a targeted temperature management (TTM) system, including a TTM module configured to provide a TTM fluid and a thermal pad configured to facilitate thermal energy transfer between the TTM fluid and a patient. The pad includes a pad portion configured for placement on the patient, a fluid delivery conduit extending away from the pad portion, where the fluid delivery conduit includes a delivery conduit connector at a proximal end thereof. The pad further includes a fluid return conduit extending away from the pad portion, where the fluid return conduit includes a return conduit connector at a proximal end thereof. The system further includes a fluid delivery line (FDL) including a fluid delivery lumen and a fluid return lumen, where the lumens extend from a proximal end to a distal end of the FDL, and where the FDL is coupled with the TTM module at the proximal end. The FDL includes an FDL hub at the distal end. The hub includes a delivery hub connector coupled with the delivery conduit connector and a return hub connector coupled with the return conduit connector. The hub also includes a locking mechanism selectively configurable between a release configuration and a lock configuration. When the locking mechanism is in the lock configuration, the locking mechanism prevents at least one of separation of the delivery conduit connector from the delivery hub connector or separation of the return conduit connector from the return hub connector. In some embodiments, the delivery conduit connector is attached to the return conduit connector.

In some embodiments, when the locking mechanism is in the lock configuration, the locking mechanism prevents separation of the delivery conduit connector from the delivery hub connector and separation the return conduit connector from the return hub connector. The locking mechanism may include a rotatable knob, and when the locking mechanism is transitioned from the release configuration to the lock configuration, the knob is rotated from a first angular position to a second angular position.

The delivery hub connector and/or the return hub connector may include a valve. Connecting the delivery conduit connector with the delivery hub connector may open the valve of the delivery hub connector and disconnecting the delivery conduit connector from the delivery hub connector may close the valve of the delivery hub connector. The valve of the delivery hub connector may be closed unless the delivery conduit connector is coupled with the delivery hub connector.

The valve may include a septum extending across a lumen of the delivery hub connector and the septum may include a slit configured to be disposed between an open configuration and a closed configuration, such that when the slit is in the open configuration, flow of TTM fluid through the delivery hub connector is allowed, and when the slit is in the closed configuration, flow of TTM fluid through the delivery hub connector is prevented.

The thermal pad may include a radio frequency identification (RFID) tag configured to provide pad identification data, and the TTM module may include an RFID sensor configured to receive pad identification data from the RFID tag. Pad identification logic stored in memory of the TTM module may be configured to alert the clinician according to an identification of the pad.

The thermal pad may include a filter in fluid communication with the fluid delivery conduit such that TTM fluid passing through the fluid delivery conduit passes through the filter and the filter may include a porous wall oriented parallel to a continuous flow path through the filter.

Further disclosed herein is a medical pad for exchanging thermal energy between a targeted temperature management (TTM) fluid and a patient. The pad includes a pad portion configured for placement on the patient; a fluid delivery conduit extending away from the pad portion, where the fluid delivery conduit includes a delivery conduit connector at a proximal end thereof; and a fluid return conduit extending away from the pad portion, where the fluid return conduit includes a return conduit connector at a proximal end thereof. The pad further includes an RFID tag configured to provide pad identification data to an RFID sensor, and the RFID tag may be attached to the pad portion.

The pad may further include a fluid containing layer configured to contain circulating TTM fluid therein and an insulation layer coupled with the fluid containing layer. The RFID tag may be disposed between the insulation layer and the fluid containing layer.

The delivery conduit connector and the return conduit connector may be attached together. The delivery conduit connector and the return conduit connector may be configured to couple with a fluid delivery line of a TTM module to establish fluid communication of the fluid delivery conduit and the fluid return conduit with the fluid delivery line.

In some embodiments of the pad, at least one of the delivery conduit connector or the return conduit connector may be configured to be locked to the fluid delivery line to prevent separation of the at least one of the delivery conduit connector or the return conduit connector from the fluid delivery line.

The pad may include a filter in fluid communication with the fluid delivery conduit such that TTM fluid passing through the fluid delivery conduit passes through the filter, and the filter may include a porous wall oriented parallel to a continuous flow path through the filter.

Further disclosed herein is a method of exchanging thermal energy with a patient. The method includes providing a targeted temperature management (TTM) module configured to circulate TTM fluid through one or more thermal pads. The TTM module includes a fluid delivery line (FDL) for transporting TTM fluid to and from the one or more thermal pads and the FDL includes an FDL hub at a distal end.

The method further includes providing a thermal pad that includes a pad portion configured for placement on the patient. The pad portion includes a layer for containing TTM fluid; a fluid delivery conduit coupled with the pad portion at a distal end of the fluid delivery conduit, where the fluid delivery conduit includes a delivery conduit connector at a proximal end thereof; and a fluid return conduit coupled with the pad portion at a distal end of the fluid return conduit, where the fluid return conduit includes a return conduit connector at a proximal end thereof.

The method further includes connecting the delivery conduit connector and the return conduit connector to the FDL hub to establish fluid communication of the fluid delivery conduit and the fluid return conduit with the FDL, actuating a locking mechanism of the FDL hub to secure the delivery conduit connector and the return conduit connector to the FDL hub, applying the pad portion to the patient, and circulating TTM fluid through the thermal pad.

In some embodiments of the method, the locking mechanism includes a knob, and actuating the locking mechanism includes rotating the knob from a first angular position to a second angular position. Actuating the locking mechanism may further include displacing the knob from an extended position to a depressed position. Rotating the knob from the first angular position to the second angular position may be performed after displacing the knob from the extended position to the depressed position. Rotating the knob from the first angular position to the second angular position may be prevented when the knob is in the extended position and rotating the knob from the second angular position to the first angular position may also be prevented when the knob is in the extended position. The knob may be biased toward the extended position.

The method may further include deactivating the locking mechanism to release the delivery conduit connector and the return conduit connector from the FDL hub, and deactivating the locking mechanism may include rotating the knob from the second angular position to the first angular position. Deactivating the locking mechanism may also include allowing to the knob to self-displace from the depressed position to the extended position.

These and other features of the concepts provided herein will become more apparent to those of skill in the art in view of the accompanying drawings and the following description, which describe particular embodiments of such concepts in greater detail.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates a targeted temperature management (TTM) system for cooling or warming a patient, in accordance with some embodiments.

FIG. 2 illustrates a hydraulic schematic of the TTM system of FIG. 1, in accordance with some embodiments.

FIG. 3 illustrates a block diagram depicting various elements of a console of the TTM module of FIG. 1, in accordance with some embodiments.

FIG. 4A is a top view of a thermal pad of the system of FIG. 1, in accordance with some embodiments.

FIG. 4B is a cross-sectional view of the pad of FIG. 4A cut along sectioning lines 4B-4B, in accordance with some embodiments.

FIG. 5A is an exploded view of the fluid delivery line hub and proximal portions of the fluid conduits of FIG. 1, in accordance with some embodiments.

FIG. 5B illustrates is a top view of the hub and the proximal portions of the fluid conduits of FIG. 5A, in accordance with some embodiments.

FIG. 5C is a top cross-sectional view of the hub and the proximal portions of the fluid conduits of FIG. 5B, in accordance with some embodiments.

FIG. 6A is a detail cross-sectional view of a portion the hub of FIG. 5B cut along sectioning lines 6A-6A with the knob disposed in the release position, in accordance with some embodiments.

FIG. 6B is the detail cross-sectional view of FIG. 6A with the knob rotated to the lock position, in accordance with some embodiments.

FIG. 6C is a detail view of FIG. 5C further illustrating the knob in the release position, in accordance with some embodiments.

FIG. 6D is the detail view of FIG. 6C further with the knob rotated to the lock position, in accordance with some embodiments.

FIG. 6E is a detail view of FIG. 5C illustrating the knob in obstruction engagement with the scallop of the conduit connector, in accordance with some embodiments.

FIG. 7A is a top view of a portion of another embodiment hub, in accordance with some embodiments.

FIG. 7B is a bottom view of the portion of hub of FIG. 7A, in accordance with some embodiments.

FIG. 7C is a detail cross-sectional view of the portion of FIG. 7A cut along sectioning lines 7C-7C with the knob disposed in the release position, in accordance with some embodiments.

FIG. 7D is a detail cross-sectional view of the portion of FIG. 7A cut along sectioning lines 7D-7D with the knob disposed in the release position, in accordance with some embodiments.

FIG. 7E is a detail cross-sectional view of FIG. 7C with the knob rotated to the lock position, in accordance with some embodiments.

FIG. 7F is a detail cross-sectional view of the portion of FIG. 7D with the knob rotated to the lock position, in accordance with some embodiments.

FIG. 8A provides an exploded perspective view of a TTM fluid filter, in accordance with some embodiments.

FIG. 8B is a cross-sectional side view of the filter of FIG. 8A, in accordance with some embodiments.

FIG. 8C is a cross-sectional detail view of the thermal contact pad of FIG. 4A incorporating the filter of FIG. 8A, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” “horizontal,” “vertical” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. The words “including,” “has,” and “having,” as used herein, including the claims, shall have the same meaning as the word “comprising.” Furthermore, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, components, functions, steps or acts are in some way inherently mutually exclusive.

The phrases “connected to” and “coupled with” refer to any form of interaction between two or more entities, including mechanical, electrical, magnetic, electromagnetic, fluid, signal, communicative (including wireless), and thermal interaction. Two components may be connected to or coupled with each other even though they are not in direct contact with each other. For example, two components may be coupled with each other through an intermediate component.

The directional terms “proximal” and “distal” are used herein to refer to opposite locations on a medical device. The proximal end of the device is defined as the end of the device closest to the end-user when the device is in use by the end-user. The distal end is the end opposite the proximal end, along the longitudinal direction of the device, or the end furthest from the end-user.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

Unless defined otherwise, all technical and scientific terms used herein have the same meaning as commonly understood by those of ordinary skill in the art.

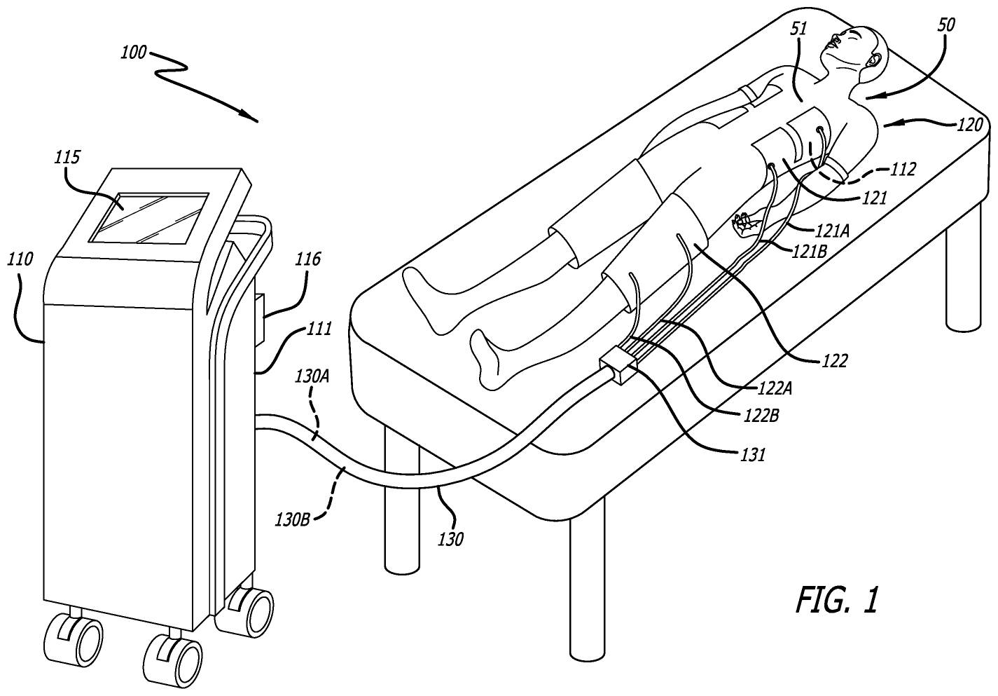

FIG. 1 illustrates a targeted temperature management (TTM) system 100 connected to a patient 50 for administering TTM therapy to the patient 50 which may include a cooling and/or warming of the patient 50, in accordance with some embodiments. The TTM system 100 includes a TTM module 110, a fluid delivery line (FDL) 130, and a thermal contact pad set 120. In the illustrated embodiment, the pad set 120 includes two thermal contact pads (pads) 121, 122. In other embodiments, the pad set 120 may include 1, 2, 3, 4, 5, 6, or more thermal contact pads. In the illustrated embodiments, the FDL 130 is configured to couple with two thermal pads. In other embodiments, the FDL 130 may be configured to couple with 1, 2, 3, 4, 5, 6, or more thermal contact pads. In some embodiments, the system 100 may include more than one FDL 130.

Each pad includes a fluid delivery conduit and a fluid return conduit (sometimes referred to generally as the fluid conduits) coupled with the FDL 130 via an FDL hub 131. The FDL 130 includes a fluid delivery lumen 130A and a fluid return lumen 130B. In the illustrated embodiment, the pad 121 includes the fluid delivery conduit 121A coupled with the FDL 130 so as to be in fluid communication with the fluid delivery lumen 130A and a fluid return conduit 121B coupled with the FDL 130 so as to be in fluid communication with the fluid return lumen 130B. Similarly, the pad 122 includes the fluid delivery conduit 122A coupled with the FDL 130 so as to be in fluid communication with the fluid delivery lumen 130A and a fluid return conduit 122B coupled with the FDL 130 so as to be in fluid communication with the fluid return lumen 130B.

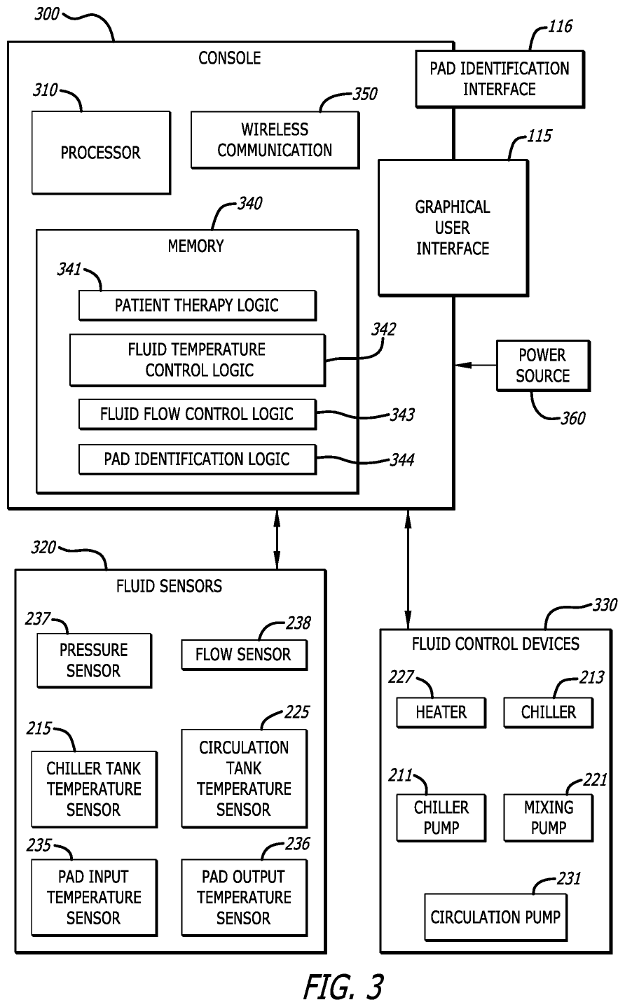

In use, the TTM module 110 prepares the TTM fluid 112 for delivery to the pad set 120 by heating or cooling the TTM fluid 112 to a defined temperature in accordance with prescribed TTM therapy parameters input by clinician via a graphical user interface 115. The TTM module 110 circulates the TTM fluid 112 between the TTM module 110 and the pad set 120 via the FDL 130. The pad set 120 is applied to the skin 51 of the patient to facilitate thermal energy exchange between the pad set 120 and the patient 50. During the TTM therapy, the TTM module 110 may continually control the temperature of the TTM fluid 112 toward a target TTM temperature. The TTM module 110 may further include a pad identification interface 116 as further described below in relation to FIG. 3

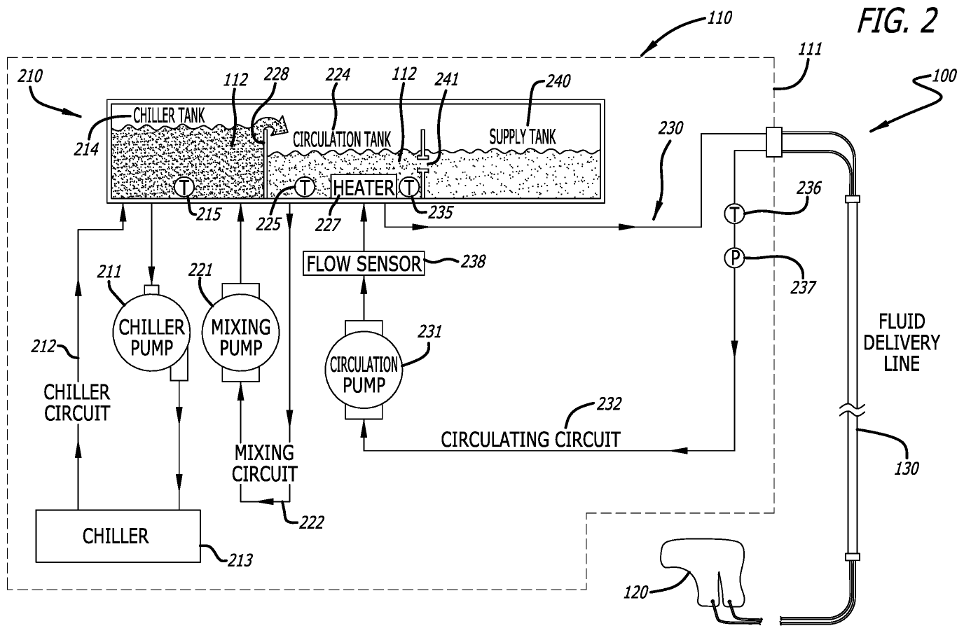

FIG. 2 illustrates a hydraulic schematic of the TTM system 100. The pad set 120 (FIG. 1) along with the corresponding fluid conduits are disposed external to the housing 111 of the TTM module 110. The TTM module includes various fluid sensors and fluid control devices to prepare and circulate the TTM fluid 112. The fluid subsystems of the TTM module may include a temperature control subsystem 210 and a circulation subsystem 230.

The temperature control subsystem 210 may include a chiller pump 211 to pump (recirculate) TTM fluid 112 through a chiller circuit 212 that includes a chiller 213 and a chiller tank 214. A temperature sensor 215 within the chiller tank 214 is configured to measure a temperature of the TTM fluid 112 within the chiller tank 214. The chiller 213 may be controlled by a temperature control logic (see FIG. 3) as further described below to establish a desired temperature of the TTM fluid 112 within chiller tank 214. In some instances, the temperature of the TTM fluid 112 within the chiller tank 214 may be less than the target temperature for the TTM therapy.

The temperature control subsystem 210 may further include a mixing pump 221 to pump TTM fluid 112 through a mixing circuit 222 that includes the chiller tank 214, a circulation tank 224, and a dam 228 disposed between the chiller tank 214 and circulation tank 224. The TTM fluid 112, when pumped by the mixing pump 221, enters the chiller tank 214 and mixes with the TTM fluid 112 within the chiller tank 214. The mixed TTM fluid 112 within the chiller tank 214 flows over the dam 228 and into the circulation tank 224. In other words, the mixing circuit 222 mixes the TTM fluid 112 within chiller tank 214 with the TTM fluid 112 within circulation tank 224 to cool the TTM fluid 112 within the circulation tank 224. A temperature sensor 225 within the circulation tank 224 measures the temperature of the TTM fluid 112 within the circulation tank 224. The temperature control logic may control the mixing pump 221 in accordance with temperature data from the temperature sensor 225 within the circulation tank 224.

The circulation tank 224 includes a heater 227 to increase to the temperature of the TTM fluid 112 within the circulation tank 224, and the heater 227 may be controlled by the temperature control logic. In summary, the temperature control logic when executed by the processor (see FIG. 3) may 1) receive temperature data from the temperature sensor 215 within the chiller tank and the temperature sensor 225 within the circulation tank 224 and 2) control the operation of the chiller 213, the chiller pump 211, the heater 227, and mixing pump 222 to establish and maintain the temperature of the TTM fluid 112 within the circulation tank 224 at the target temperature for the TTM therapy.

The circulation subsystem 230 includes a circulation pump 213 to pull TTM fluid 112 from the circulation tank 224 and through a circulating circuit 232 that includes the pad set 120 located upstream of the circulation pump 213. The circulating circuit 232 also includes a pressure sensor 237 to represent a pressure of the TTM fluid 112 within the pad set 120. The circulating circuit 232 includes a temperature sensor 235 within the circulation tank 224 to represent the temperature of the TTM fluid 112 entering the pad set 120 and a temperature sensor 236 to represent the temperature of the TTM fluid exiting the pad set 120. A flow meter 238 is disposed downstream of the circulation pump 213 to measure the flow rate of TTM fluid 112 through the circulating circuit 232 before the TTM fluid 112 re-enters that the circulation tank 224.

In use, the circulation tank 224, which may be vented to atmosphere, is located below (i.e., at a lower elevation than) the pad set 120 so that a pressure within the pad set 120 is less than atmospheric pressure (i.e., negative) when TTM fluid flow through the circulating circuit 232 is stopped. The pad set 120 is also placed upstream of the circulation pump 231 to further establish a negative pressure within the pad set 120 when the circulation pump 213 is operating. The fluid flow control logic (see FIG. 3) may control the operation of the circulation pump 213 to establish and maintain a desired negative pressure within the pad set 120. A supply tank 240 provides TTM fluid 112 to the circulation tank 224 via a port 241 to maintain a defined volume of TTM fluid 112 within the circulation tank 224.

FIG. 3 illustrates a block diagram depicting various elements of the TTM module 110 of FIG. 1, in accordance with some embodiments. The TTM module 110 includes a console 300 including a processor 310 and memory 340 including non-transitory, computer-readable medium. Logic modules stored in the memory 340 include patient therapy logic 341, fluid temperature control logic 342, fluid flow control logic 343, and pad identification logic 344. The logic modules when executed by the processor 310 define the operations and functionality of the TTM Module 110.

Illustrated in the block diagram of FIG. 3 are fluid sensors 320 as described above in relation to FIG. 2. Each of the fluid sensors 320 are coupled with the console 300 so that data from the fluid sensors 320 may be utilized in the performance of TTM module operations. Fluid control devices 330 are also illustrated in FIG. 3 as coupled with the console 300. As such, logic modules may control the operation of the fluid control devices 330 as further described below.

The patient therapy logic 341 may receive input from the clinician via the GUI 115 to establish operating parameters in accordance with a prescribed TTM therapy. Operating parameters may include a target temperature for the TTM fluid 112 and/or a thermal energy exchange rate which may include a time-based target temperature profile. In some embodiments, the fluid temperature control logic 342 may define other fluid temperatures of the TTM fluid 112 within the TTM module 110, such a target temperature for the TTM fluid 112 within the chiller tank 214, for example.

The fluid temperature control logic 342 may perform operations to establish and maintain a temperature of the TTM fluid 112 delivered to the pad set 120 in accordance with the predefined target temperature. One temperature control operation may include chilling the TTM fluid 112 within the chiller tank 214. The fluid temperature control logic 342 may utilize temperature data from the chiller tank temperature sensor 215 to control the operation of the chiller 213 to establish and maintain a temperature of the TTM fluid 112 within the chiller tank 214.

Another temperature control operation may include cooling the TTM fluid 112 within the circulation tank 224. The fluid temperature control logic 342 may utilize temperature data from the circulation tank temperature sensor 225 to control the operation of the mixing pump 221 to decrease the temperature of the TTM fluid 112 within the circulation tank 224 by mixing TTM fluid 112 from the chiller tank 214 with TTM fluid 112 within circulation tank 224.

Still another temperature control operation may include warming the TTM fluid 112 within the circulation tank 224. The fluid temperature control logic 342 may utilize temperature data from the circulation tank temperature sensor 225 to control the operation of the heater 227 to increase the temperature of the TTM fluid 112 within the circulation tank 224.

The fluid flow control logic 343 may control the operation of the circulation pump 231. As a thermal energy exchange rate is at least partially defined by the flow rate of the TTM fluid 112 through the pad set 120, the fluid flow control logic 343 may, in some embodiments, control the operation of the circulation pump 231 in accordance with a defined thermal energy exchange rate for the TTM therapy.

The console 300 may include or be coupled with a wireless communication module 350 to facilitate wireless communication with external devices. A power source 360 provides electrical power to the console 300.

The identification interface 116 may be coupled with the console 300 and provide pad identification data to the pad identification logic 344. The pad identification logic 344 may be configured so that, when executed by the processor 310, pad identification logic 344 may alert the clinician as to the identification of each thermal pad of the pad set 120. In an embodiment, the pad identification logic 344 may alert the clinician that one or more pads were not manufactured by a defined set of manufacturers. For example, if the identification interface 116 does not receive any pad identification data, the pad identification logic 344 may alert the clinician accordingly.

In some embodiments, the pad identification interface 116 may be configured to wirelessly receive pad identification data from the pad set 120. In the illustrated embodiment, the pad identification interface 116 may include a radio frequency identification (RFID) sensor configured to receive pad identification data from one or more RFID tags coupled with any or all pads of the pad set 120. In some instances, an air-in-line detector may identify air (or “bubbles”) in the TTM fluid 112. For example, an air-in-line detector may detect air along either of the fluid delivery conduit 121A or the fluid return conduit 121B. Upon detection of air in the TTM fluid 112, an alert may be generated for the clinician that includes an identifier derived from an RFID tag of the pad 121. Thus, the clinician would be alerted to the presence of air in the TTM fluid 112 flowing through (or which has passed through) a specific pad 121. As a result, the clinician may check the connections for that particular pad 121. In other embodiments, other tags or means for obtaining an identifier of a particular pad 121 may be utilized in place of a RFID tag.

In some embodiments, the identification data may include a set of identification parameters (e.g., pad size), and the memory may include a corresponding set of identification parameters. An operation of the pad identification logic 344 may include comparing an identification parameter of the identification data with a corresponding identification parameter stored in memory, and the identification logic may be configured to modify the operation of the system in accordance with a result of the comparison.

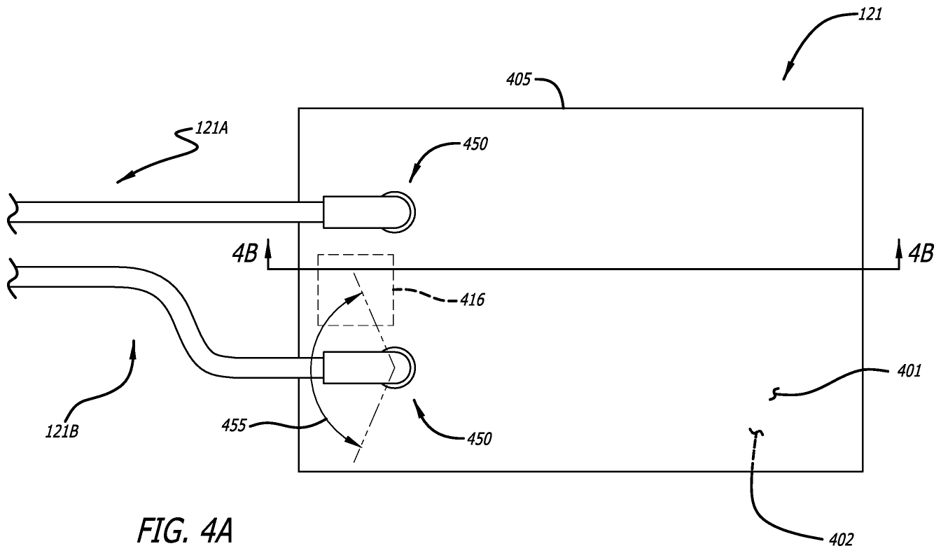

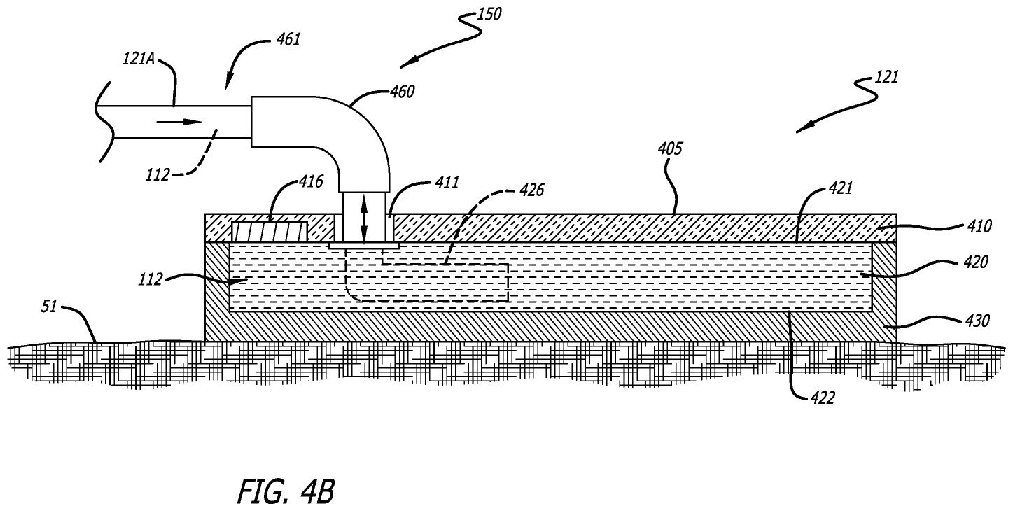

FIG. 4A shows a top view of the thermal contact pad 121. While the description that follows describes features, components and details of the pad 121, the description that follows may equally apply to any and all other thermal contact pads of the pad set 120. The fluid delivery conduit 121A and the fluid return conduit 121B extend away from the joints 450, in accordance with some embodiments. As illustrated, the joints 450 may provide for a rotatable connection between fluid delivery conduit 121A and the fluid return conduit 121B and a pad portion 405 of the pad 121. The rotatable connection may provide for the fluid conduit to rotate through an angle 455 ranging up to about 90 degrees, 180 degrees, 360 degrees, or more. In some embodiments, the joint 450 may define a fixed rotatable connection, i.e., the joint may allow rotation but not separation. In other embodiments, the joint 450 may define a pre-assembled rotatable connection that allows rotation and separation by the clinician. The pad 121 may include an RFID tag 416 coupled thereto for providing pad identification data as further described below.

FIG. 4B shows a cross-sectional side view of the pad portion 405 of the thermal contact pad 121 of FIG. 4A in contact with the patient 50, in accordance with some embodiments. The pad 121 may include multiple layers to provide multiple functions of the pad 121. A fluid containing layer 420 is fluidly coupled with the fluid delivery conduit 121A via the joint 450 to facilitate circulation of the TTM fluid 112 within the fluid containing layer 420. Similarly, (although not shown in FIG. 4B) the fluid containing layer 420 is fluidly coupled with the fluid return conduit 121B via the joint 450. The fluid containing layer 420 having TTM fluid 112 circulating therein defines a heat sink or a heat source for the patient 50 in accordance with a temperature of the TTM fluid 112. The fluid delivery conduit 121A may also be coupled with an internal fluid conduit 426 of the fluid containing layer 420 so that TTM fluid 112 entering the fluid containing layer 420 passes through the internal fluid conduit 426.

The pad 121 may include a thermal conduction layer 430 disposed between the fluid containing layer 420 and the patient 50. The thermal conduction layer 430 is configured to facilitate thermal energy transfer between the fluid containing layer 420 and the patient 50. The thermal conduction layer 430 may be attached to the thermal conduction layer 430 along a bottom surface 421 of the fluid containing layer 420. The thermal conduction layer 430 may be conformable to provide for intimate contact with the patient 50. In other words, thermal conduction layer 430 may conform to a contour of the patient 50 to inhibit the presence space or air pockets between the thermal conduction layer 430 and the patient 50.

The pad 121 may include an insulation layer 410 disposed on the top side of the fluid containing layer 420. The insulation layer 410 is configured to inhibit thermal energy transfer between the fluid containing layer 420 and the environment. The insulation layer 410 may be attached to the fluid containing layer 420 along a top surface 422 of the fluid containing layer 420. In some embodiments, the insulation layer 410 may include one or more openings 411 extending through the insulation layer 410 to provide for coupling of the fluid delivery conduit 121A and fluid return conduit 121B with the fluid containing layer 420.

The joint 450 may include an elbow 460 to change the orientation of the fluid delivery conduit 121A. As shown, the orientation of 130 is shifted from an orientation that is perpendicular to the pad 121 to an orientation that is substantially parallel to the pad 121. The elbow 460 also establishes an orientation of a distal portion 461 of the fluid delivery conduit 121A to be substantially parallel to the pad 121 and/or the fluid containing layer 420.

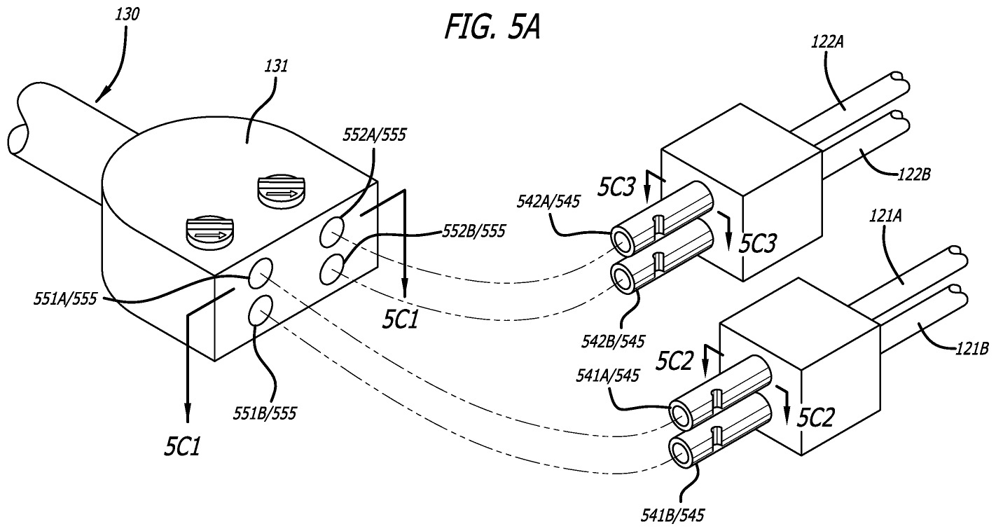

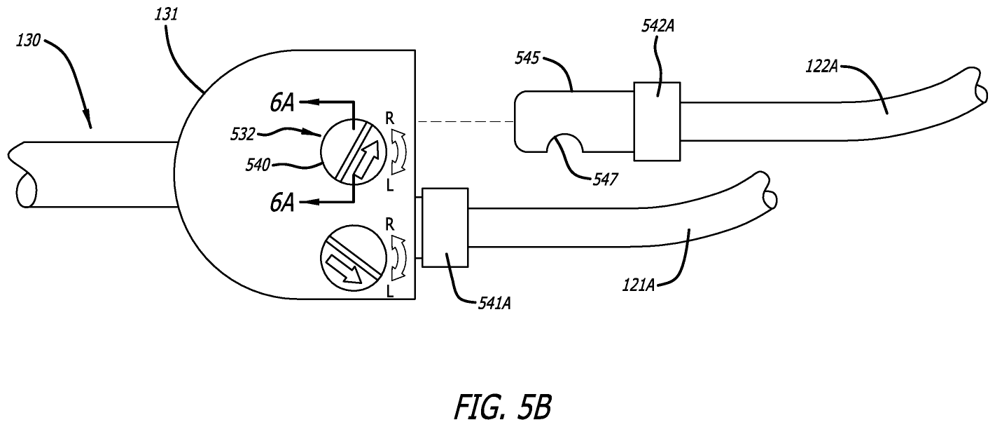

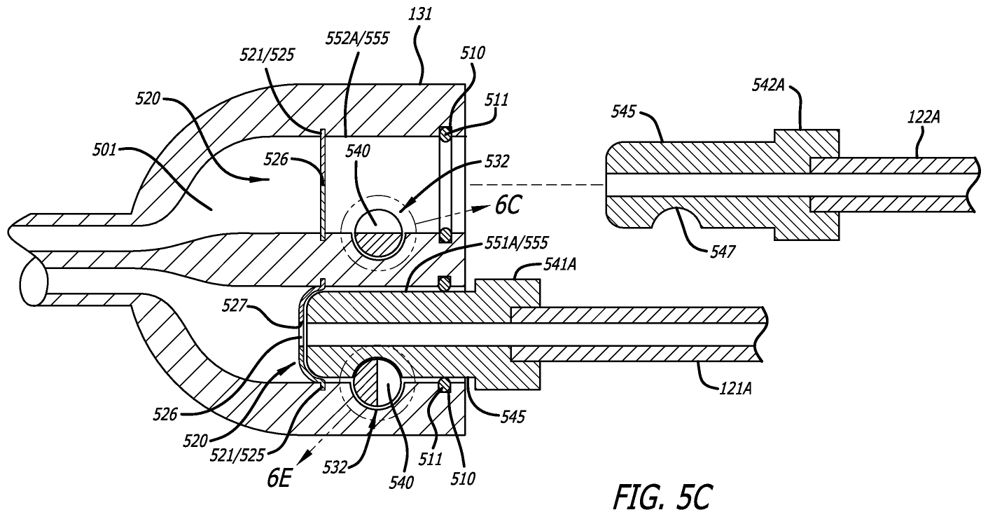

The RFID tag 416 may be disposed between layers of the pad 121 such as between the insulation layer 410 and the fluid containing layer 420. In some embodiments, the RFID tag 416 may be located between any two layers or on the top side 410 of the pad 121. In other embodiments, the RFID tag 416 may be embedded within a layer, such as the insulation layer 410, for example. In still other embodiments, the RFID tag may be attached to one of the fluid delivery conduit 121A, the fluid return conduit 121B, the delivery conduit connector 541A or the return conduit connector 541B (see FIG. 5A below).

FIG. 5A illustrates an end perspective view of the hub 131 and end perspective views of the conduit connectors 541A-542B showing how the conduit connectors 541A-542B may be connected to the hub 131. As shown, the delivery conduit connector 541A is attached to the return conduit connector 541B. Similarly, the delivery conduit connector 542A is attached to the return conduit connector 542B. The attachment of the connectors together may facilitate simplicity when connecting the connectors to the hub 131. For example, the clinician may couple the delivery conduit connector 541A and the return conduit connector 541B to the hub 131 via a single motion or step.

The hub 131 includes hub connectors that correspond to the conduit connectors 541A-542B. In the illustrated embodiment, the hub 131 includes hub connectors 551A, 551B, 552A, and 552B which may be integral to the hub 131. The conduit connectors 541A, 541B, 542A and 542B are coupled with the fluid delivery conduits 121A, 121B, 122A and 122B, respectively. In the illustrated embodiment, upon connection of the pad set 120 to the FDL 130, the conduit connectors may be coupled with the hub connectors such that 541A is coupled with 551A, 541B is coupled with 551B, 542A is coupled with 552A, and 542B is coupled with 552B. In another embodiment, the conduit connectors may be coupled with the hub connectors such that 541A is coupled with 551B, 541B is coupled with 551A, 542A is coupled with 552B, and 542B is coupled with 552A. In still other embodiments, other arrangements of the connectors are also possible.

In some embodiments, the conduit connectors designated as “A” may functionally correspond with (i.e., couple with) the hub connectors designated as “A.” Similarly, the conduit connectors designated as “B” may functionally correspond with (i.e., couple with) the hub connectors designated as “B.” In some embodiments, an “A” designated conduit connector may only couple with an “A” designated hub connector and a “B” designated conduit connector may only couple with a “B” designated hub connector.

As shown, the conduit connector and the hub connector may define a male-female engagement. As such, each of the conduit connectors 541A-542B includes a post 545 and each of the hub connectors 551A-551B includes an opening 555 such that during the connection process the post 545 is inserted into the opening 555. The post 545 may include a scallop 547 as further described below.

FIGS. 5B-5C illustrate features and details of the conduit connectors 541A-542B and the hub connectors 551A-552B. For simplicity in description, the conduit connectors 541A-542B may be singularly referred to as “the conduit connector.” As such, unless otherwise specifically stated, the description that follows in reference to the conduit connector applies equally as well to each of the conduit connectors 541A-542B. Similarly, the hub connectors 551A-552B may be singularly referred to as “the hub connector,” and unless otherwise specifically stated, the description that follows in reference to the hub connector applies equally as well to each of the hub connectors 551A-552B.

FIG. 5B illustrates a top view of the hub 131, the conduit connector 541A and conduit connector 542A. Hidden within the hub 131 are the hub connectors 551A-552B. Also hidden beneath the conduit connectors 541A, 542A are conduits connectors 541B, 542B. FIG. 5C illustrates the conduit connector 541A in a state of connection with the hub connector 551A and further illustrates the conduit connector 542A in a state of disconnection with the hub connector 552A.

In the illustrated embodiment, hub 131 may include a retention mechanism for each thermal pad 121, 122 such as the exemplary retention mechanism 532. As illustrated, the hub 131 includes the retention mechanism 532 to retain the conduit connectors 541A, 541B and further includes another retention mechanism 532 to retain the conduit connectors 542A, 542B. For simplicity in description, the retention mechanisms may be singularly referred to as the retention mechanism 532. As such, unless otherwise specifically stated, the description that follows in reference the retention mechanism applies equally as well to all retention mechanisms.

The retention mechanism 532 may be configured for selective disposition in a lock state and a release state. In the release state, connection of the conduit connector to the hub connector may be allowed. Similarly, in the release state, disconnection of the conduit connector from the hub connector may be allowed. Conversely, in the lock state, connection of the conduit connector to the hub connector may be prevented. Similarly, in the lock state, disconnection of the conduit connector from the hub connector may be prevented. In some embodiments, in the lock state, connection of the conduit connector to the hub connector may be allowed.

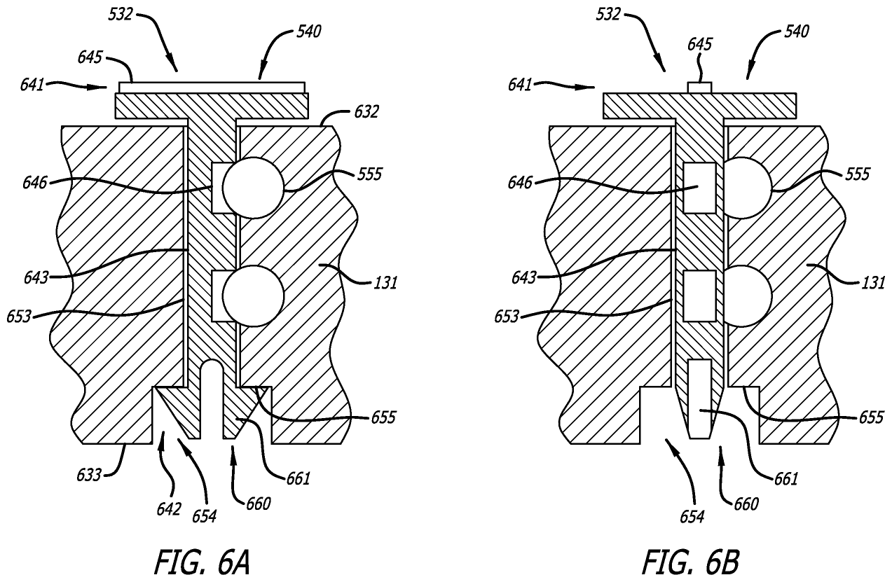

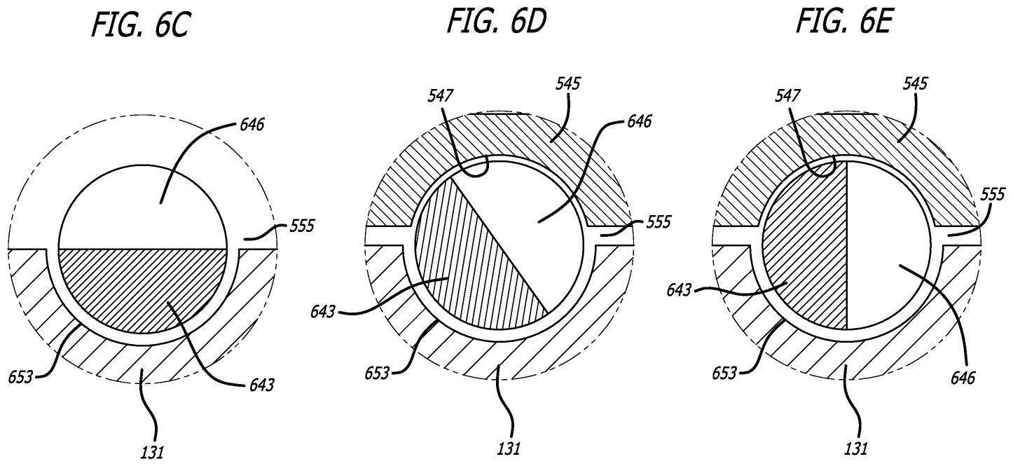

In some embodiments, selective disposition of the retention mechanism 532 between the lock state and the release state may correspond with rotation of a knob 540. More specifically, the knob 540 may be disposed in a first an angular position in accordance with the release state of the retention mechanism 532. Alternatively, the knob 540 may be disposed in second an angular position in accordance with the lock state of the retention mechanism 532. Further description of the retention mechanism 532 follows below in relation to FIGS. 6A-6E.

FIG. 5C illustrates a cross-sectional top view of the hub 131 cut along sectioning lines 5C1-5C1, a cross-sectional top view of the conduit connector 541A cut along sectioning lines 5C2-5C2, and a cross-sectional top view of the conduit connector 542A cut along sectioning lines 5C3-5C3. FIG. 5C illustrates the conduit connector 541A in a state of connection with the hub connector 551A and illustrates the conduit connector 542A in a state of disconnection with the hub connector 552A.

The hub connector may include a valve 520 disposed in line with the hub connector so that TTM fluid 112 passing through the hub connector passes through the valve 520. The valve 520 may be integrated into the hub connector. The valve 520 may be actuated in conjunction with the connecting process of the hub connector. For example, the valve 520 integrated into the hub connector 551A may be closed to prevent flow of fluid (e.g., TTM fluid 112 or air) through the hub connector 551A unless a corresponding connector (e.g., the conduit connector 541A) is coupled thereto. Similarly, the valve 520 may be open to allow flow of fluid through the hub connector when the conduit connector is coupled therewith. For example, flow of fluid through the hub connector 551A is automatically allowed when the conduit connector 541A is coupled with the hub connector 551A and automatically disallowed when the delivery conduit connector 541A is decoupled from (or not coupled with) the hub connector 551A.

The valve 520 may include a deflectable valve member 521. The valve 520 may be configured so that the valve 520 is disposed in an open configuration when the deflectable valve member 521 is deflected. Conversely, the valve 520 may be disposed in a closed configuration when the deflectable valve member 521 is not deflected. In some embodiments, the deflectable valve member 521 may be deflected via contact with the conduit connector (e.g., via connection of the conduit connector 541A with the hub connector 551A).

In the illustrated embodiment, the deflectable valve member 521 is a septum 525 disposed across a lumen 501 of the hub connector. In other embodiments, the deflectable valve member 521 may be a flexible disk, a displaceable sealing member, or any other suitable deflectable or displaceable component or system of components configured to selectively allow and prevent/inhibit flow of fluid (e.g., TTM fluid 112 or air) through the hub connector in response to connection to and disconnection from of the conduit connector, respectively.

The septum 525 may include a slit 526. The septum 525 may be configured so that TTM fluid 112 passing through the hub connector passes through the slit 526. As such, the valve 520 may be closed when the slit 526 is closed, and the valve 520 may be open when the slit 526 is open. By way of example, as shown in FIG. 5C, the conduit connector 542A is disconnected from the hub connector 552A and as such, the slit 526 of septum 525 disposed in the hub connector 552A is in a closed state. By way of further example, as shown in FIG. 5C, the conduit connector 541A is connected to the hub connector 551A. As shown, a tip of the conduit connector 541A has deflected the septum 525 disposed in hub connector 551A. As such, the slit 526 of septum 525 disposed in the hub connector 551A is in an open state.

In some embodiments, the valve 520 may be actuatable via a retention mechanism such as the retention mechanism 532 In such an instance, the valve 520 may be configured to transition from the closed state to the open state when the retention mechanism 532 is transitioned from the release state to the lock state and vice versa.

The hub connector may include a sealing member 511 disposed within an annular grove 510. The groove 510, the sealing member 511, and the post 545 may be correspondingly sized to define a compression of the sealing member 511, thereby establishing a fluid seal between the hub connector and the conduit connector. In some embodiments, the hub connector may be configured so that during the connection process with the conduit connector, the seal is established before the valve 520 is opened.

In some embodiments, the septum 525 may be configured so that the slit 526 is closed when septum 525 is in a free state, i.e., when no external forces are acting on the septum 525. In other embodiments, the septum 525 may be configured so that the slit 526 is open when septum 525 is in a free state. In such an embodiment, external forces exerted on the septum 525 when the septum 525 is installed in the hub connector may close the slit 526.

In some embodiments, septum 525 may be manufactured via an injection molding process. In one embodiment, the slit 526 may be molded into the septum 525 in a normally open state. In another embodiment, the septum 525 may be molded without the slit 526. In such an embodiment, the slit 526 may be formed in the septum 525 via a cutting process after molding, so that the slit 526 is in a normally closed state. In some embodiments, a lubricant 527 may applied to the slit 526 to prevent or inhibit re-healing of the slit 526. Re-healing of the slit 526 may prevent the slit 526 from opening to allow flow of TTM fluid 112 therethrough during use. Whether the septum 525 is formed with the slit 526 in the normally open or the normally closed state, external forces (e.g., radially inward directed forces) exerted on the septum 525 by the hub connector may at least partially define closure of the slit 526 and thereby closure of the valve 520.

In some embodiments, the valve 520 may be actuatable via fluid pressure exerted on the septum 525. For example, the valve 520 within the hub connector, may remain closed unless a pressure exceeding a first pressure magnitude is exerted on the septum 525 in the normal direction of TTM fluid flow through the hub connector, e.g., from the FDL 130 toward the pad 121 in the case of the hub connector 551A. In a further example, the valve 520 within the hub connector 551A, may remain closed unless a pressure exceeding a second pressure magnitude is exerted on the septum 525 in the opposite direction of the TTM fluid flow through the connector 551A, i.e., from the pad 121 toward the FDL 130. In such an embodiment, the second pressure magnitude may be greater than the first pressure magnitude. In other embodiments, the second pressure magnitude may be less than the first pressure magnitude.

FIGS. 6A-6E illustrate features and components of the retention mechanism 532. FIG. 6A is a cross-sectional detail view of the hub 131 cut along sectioning lines 6A-6A illustrating the retention mechanism 532. Shown are the openings 555 for each hub connector 552A, 552B within which the posts 445 of the respective conduit connectors 542A, 542B may be inserted. The retention mechanism 532 may be configured to prevent separation of the conduit connector from the hub connector by retaining the post 545 within the opening 555 of the hub connector.

FIG. 6A is a detailed cross-sectional view of a portion the hub 131 of FIG. 5B cut along sectioning lines 6A-6A with the knob 540 disposed in the release position, and FIG. 6B is the detail cross-sectional view of FIG. 6A with the knob 540 rotated to the lock position. FIG. 6C is a detail view of a portion of FIG. 5C illustrating a cross-sectional top view of the retention mechanism 532 in the release state. Similarly, FIG. 6D is the detail cross-sectional view of a FIG. 6C illustrating the retention mechanism 532 in the lock state. As stated above, the retention mechanism 532 includes the knob 540. The knob 540 is includes a cylindrical rod 643 extending from a handle 645 at a top end 641 to a bottom end 642 of the knob 540. The rod 643 is disposed within a corresponding cylindrical hole 653 extending from a top side 632 to a bottom side 633 of the hub 131. The hole 653 may include a recess 654 defining an annular ledge 655.

The knob 540 may include a snap-fit retaining mechanism 660 including as least one deflectable member 661 which may be configured to deflect inward toward a center of the rod 643. The deflectable member 661 may include a hook 565 configured to overlappingly engage the ledge 655 in a non-deflected state. In use, the knob 540 may be assembled with the hub 131 by inserting the rod 643 through the hole 653 during which the deflectable member 661 is deflected inward until the rod 643 is inserted sufficiently to dispose the deflectable member 661 within the recess 654. Once disposed within the recess, the deflectable member 661 self-deflects outward so that the hook 565 overlaps the ledge 655 thereby retaining the knob 540 within the hole 653.

As stated above, the retention mechanism 532 is configured to selectively retain the post 545 (FIG. 5C) within the opening 555. In the illustrated embodiment, the hub 131 and the knob 540 include interacting features to define the retention mechanism 532. The description below describes the details and functionally of the retention mechanism 532.

As shown, the cylindrical hole 653 is oriented orthogonal to the opening 555. The cylindrical hole 653 is positioned with respect to the opening such that the cylindrical hole 653 partially interferes with the opening 555. The cylindrical rod 643 includes a notch 646 disposed in longitudinal alignment with the opening 555. In FIGS. 6A and 6C, the notch 646 is disposed in angular alignment with the opening 555 so that the opening is unobstructed. In other words, when the retention mechanism 532 is disposed in the release state, the knob 540 is rotationally positioned so that the notch 646 is angularly aligned with the opening 555. Conversely, in FIGS. 6B and 6D, the notch 646 is disposed in angular misalignment with the opening 555 so that the opening is partially obstructed by the rod 643. In other words, when the retention mechanism 532 is disposed in the lock state, the knob 540 is rotationally positioned so that the notch 646 is angularly misaligned with the opening 555.

FIG. 6E is a detail cross-sectional view of FIG. 5C illustrating the rod 643 in obstructive engagement with the scallop 547 of the post 545. FIG. 6E is a cross-section detail view of the hub 131 showing the post 545 of the conduit connector 445 disposed within the opening 555 (see FIG. 5C). Similar to FIG. 6D, the notch 646 is disposed in angular misalignment with the opening 555 so that the opening is partially obstructed by the rod 643 consistent with the retention mechanism 532 disposed in the lock state. As shown, the scallop 547 of the post 545 is disposed in obstructing engagement with the rod 643, thereby preventing disconnection of the conduit connector from the hub connector.

As may be appreciated by one of ordinary skill, the retention mechanism 532 as shown and described is just one example of a retention mechanism configured to selectively allow and prevent disconnection of the conduit connector from the hub connector. It is to be understood that other embodiments of the retention mechanism 532 including components and features other than or in addition to the opening 555, the post 545, the scallop 547, the knob 540, and the hub 131 may be employed to facilitate selective securement of the connection between the conduit connector and the hub connector including embodiments that incorporate a rotating member, and therefore such other embodiments are included in this disclosure.

In use, the knob 540 may be initially disposed in the release position. With the knob 540 in the release position, the clinician may connect the conduit connector to the hub connector including inserting the post 545 into the opening 555. With the conduit connector coupled with the hub connector, the clinician may rotate the knob 540 from the release position to the lock position thereby preventing disconnection of the conduit connector from the hub connector. At a later time, the clinician may rotate the knob 540 from the lock position to the release position thereby allowing disconnection of the conduit connector from the hub connector. Thereafter, the clinician may decouple the conduit connector from the hub connector.

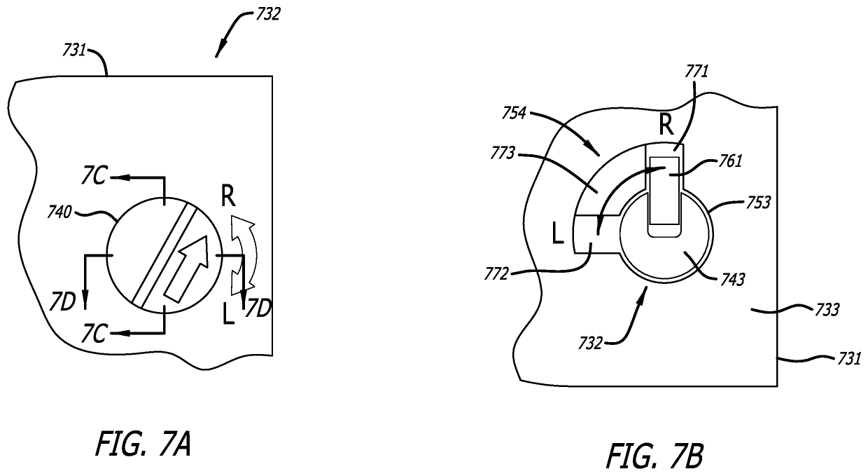

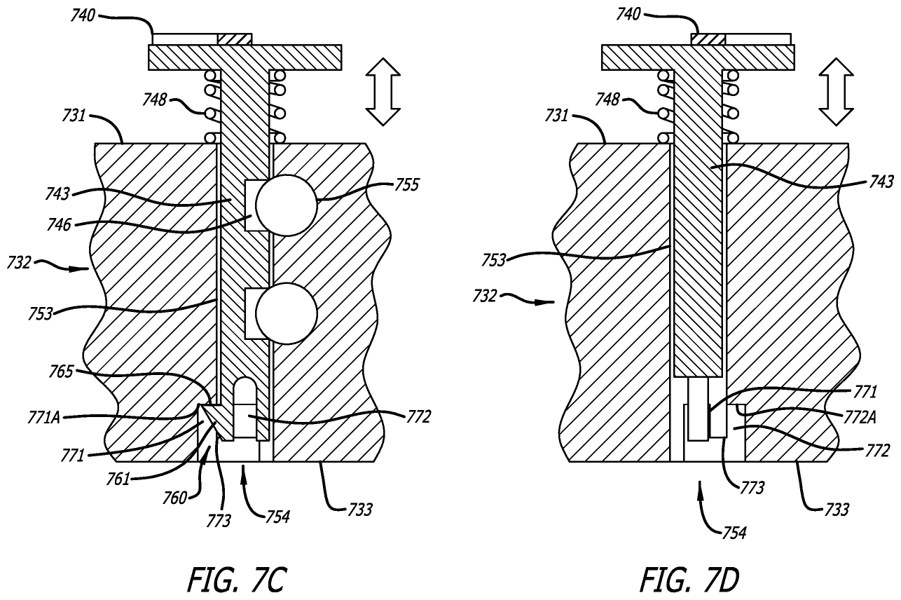

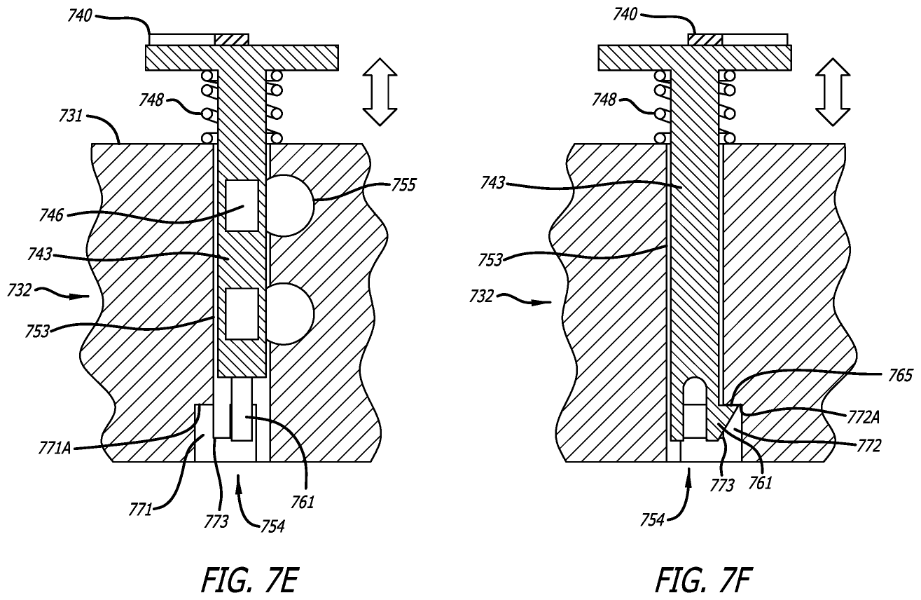

FIGS. 7A-7F illustrate another embodiment of a retention mechanism 732 that can, in certain respects, resemble components of the retention mechanism 532 described in connection with FIGS. 5A-6E. As such, the retention mechanism 732 may be incorporated into the FDL 130 of the system 100. It will be appreciated that all the illustrated embodiments may have analogous features. Accordingly, like features are designated with like reference numerals, beginning with a leading digit of “7.” For instance, the knob is designated as “540” in FIGS. 5A-6E, and an analogous knob is designated as “740” in FIGS. 7A-7F. Relevant disclosure set forth above regarding similarly identified features thus may not be repeated hereafter. Moreover, specific features of the retention mechanism 532 and related components shown in FIGS. 5A-6E may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the retention mechanism 732 of FIGS. 7A-7F. Any suitable combination of the features, and variations of the same, described with respect to the retention mechanism 532 and components illustrated in FIGS. 5A-6E can be employed with the retention mechanism 732 and components of FIG. 7A-7F, and vice versa.

FIG. 7A is a top view of a portion of the hub 731 illustrating the retention mechanism 732 including a knob 740 rotatable between a release “R” position and a lock “L” position. FIG. 7B is a bottom view the portion of the hub 731 of FIG. 7A further illustrating the retention mechanism 732. With reference to FIG. 7B, the cylindrical rod 743 of the knob 740 is disposed within the opening 753, and the cylindrical rod 743 is rotatable along with the knob 740 between a release position and a lock position. The recess 754 includes a release slot 771 within which the deflectable member 761 may be disposed when the knob 740 is in the release position. Similarly, the recess 754 includes a lock slot 772, angularly offset from the release slot 771, within which the deflectable member 761 may be disposed when the knob 740 is in the lock position. A transition ledge 773 is disposed between the release slot 771 and lock slot 772.

FIG. 7C is a cross-sectional detail view of the retention mechanism 732 cut along sectioning lines 7C-7C of FIG. 7A, and FIG. 7D is a cross-sectional detail view of the retention mechanism 732 cut along sectioning lines 7D-7D of FIG. 7A, where the sectioning lines 7D-7D are orthogonal to the sectioning lines 7C-7C. FIGS. 7A and 7B illustrate the knob 740 in the release position. As shown in FIG. 7C, the rod 743 includes a notch 746 disposed in angular alignment with the opening 755 so that the rod 743 does not obstruct the opening 755. The retention mechanism 732 includes a biasing member 748 (e.g., a coil spring) defining a biasing longitudinal force on the knob 740 in the extended direction.

With reference to FIGS. 7C and 7D, the knob 740 may include a snap-fit retaining mechanism 760 including as least one deflectable member 761 which may be configured to deflect inward toward a center of the rod 743. The deflectable member 761 may include a hook 765 configured to overlappingly engage one or more ledges in a non-deflected state as described below. The knob 740 may be assembled with the hub 731 by inserting the rod 743 through the hole 753 during which the deflectable member 761 is deflected inward until the rod 743 is inserted sufficiently to dispose the deflectable member 761 within the recess 754. Once disposed within the recess 754, the deflectable member 761 may self-deflect outward so that the hook 765 may overlap the ledges, thereby retaining the knob 740 within the hole 753. The views of FIGS. 7C and 7D illustrate the bottom-side 733 of the hub 731.

As stated above and with reference to the FIG. 7B, with the knob 740 in the release position, the deflectable member 761 may be disposed within the slot 771 thereby preventing rotation of the knob 740 away from the release position. With the knob 740 in the release position, the hook 765 is in overlapping engagement with the ledge 771A of the release slot 771, and the biasing member 748 may cause the hook 765 to abut the ledge 771A.

FIGS. 7E and 7F are analogous to the FIGS. 7C and 7D, respectively, except the knob 740 is rotated to the lock position. As shown in FIG. 7E the rod 743 is rotated so that the slot 746 is not aligned with the opening 755. Hence, the rod 743 partially obstructs the opening 755. As shown in FIG. 7F, the deflectable member 761 is disposed in the slot 772 thereby preventing the knob 740 from rotating away from the lock position. With the deflectable member 761 disposed in the slot 772, the biasing member 748 may cause the hook 765 to abut the ledge 772A.

In use, the knob 740 may be initially disposed in the release position. With the knob 740 in the release position, the clinician may connect the conduit connector to the hub connector including inserting the post 545 (see FIG. 5C) into the opening 755. With the conduit connector coupled with the hub connector, the clinician may depress the knob 740 to displace the deflectable member 761 out of the slot 771 and rotate the knob 740 from the release position to the lock position. While the knob 740 is disposed between the release position and the lock position during rotation, the hook 765 of the deflectable member 761 is disposed in overlapping engagement with the transition ledge 773. When the hook 765 is in overlapping engagement with the transition ledge 773, the knob is prevented from longitudinally displacing away from the depressed position to the extended position. With the knob 740 in the lock position, the clinician may allow the knob 740 to self-extend from the depressed state to the extended state causing the deflectable member 761 to enter into the lock slot 772 thereby preventing disconnection of the conduit connector from the hub connector. At a later time, the clinician may depress the knob 740 and then rotate the knob 740 from the lock position to the release position thereby allowing disconnection of the conduit connector from the hub connector. Thereafter, the clinician may decouple the conduit connector from the hub connector.

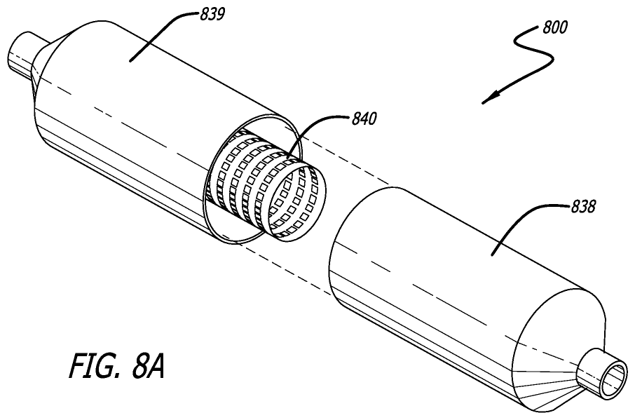

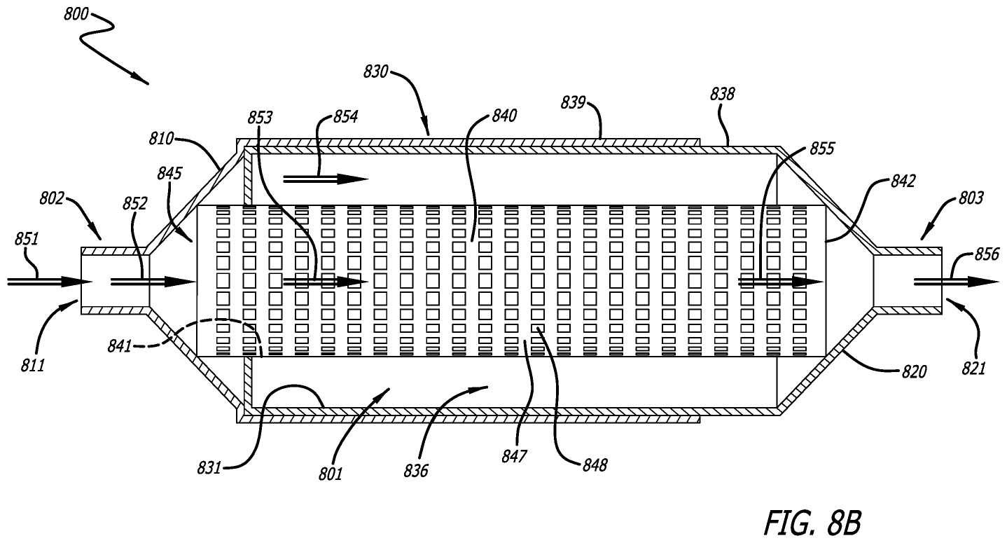

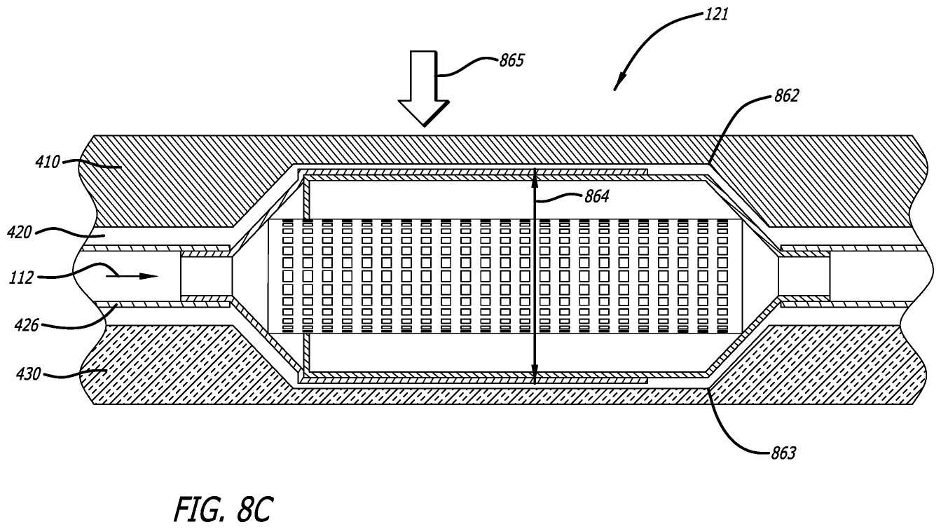

FIGS. 8A and 8B show a filter 800 that may be included with the TTM system 100. The filter 800 may be disposed in line with a TTM fluid flow path of the TTM system 100 so that the circulating TTM fluid 112 flows through the filter 800. The filter 800 may be configured to remove (i.e., filter out) material/particles having a size of 0.2 microns or larger from the TTM fluid 112 without causing a flow restriction of the TTM fluid 112.

The filter 800 includes a longitudinal shape having a flow path 801 extending from a first end 802 to a second end 803. The filter 800 includes a diffuser 810 adjacent the first end 802, a nozzle adjacent 820 the second end 803, and a body 830 extending between the diffuser 810 and the nozzle 820. Along the diffuser 810, a cross-sectional flow area of the filter 800 expands from an inlet flow area 811 to a body flow area 831 and along the nozzle 820, the cross-sectional flow area of the filter 800 contracts from the body flow area 831 to an outlet flow area 821. In some embodiments, the inlet flow area 811 and the outlet flow area 821 may be substantially equal.

In some embodiments, the body flow area 831 may be constant along the body 830. In other embodiments, the body flow area 831 may vary along a length of the body 830 such that the body flow area 831 is greater or less along middle portion of the body 830 than at the ends of the body 830. In some embodiments, the body flow area 831 may be circular.

The filter 800 includes an inner tube 840 disposed within the body 830 extending along the length of body 830. The inner tube 840 may be coupled with the diffuser 810 at a first inner tube end 841 so that TTM fluid 112 entering the filter 800 at the first end 802 also enters the inner tube 840 at the first inner tube end 841. The inner tube 840 may be coupled with the nozzle 820 at a second inner tube end 842 so that TTM fluid 112 exiting the filter 800 at the second end 803 also exits the inner tube 840 at the second inner tube end 842.

The inner tube 840 includes an inner tube flow area 845 extending the length of the inner tube 840. The inner tube flow area 845 may be greater than the inlet flow area 811 and/or the outlet flow area 821. The inner tube flow area 845 may be constant along the length of the inner tube 840. In some embodiments, the inner tube flow area 845 may vary along the length of the inner tube 840. In some embodiments, the inner tube 840 may include a circular cross section. The inner tube 840 and the body 830 may be configured so that the body flow area 831 includes a combination of the inner tube flow area 845 and an annular flow area 836.

The inner tube 840 includes a porous a circumferential wall 847. The porous wall 847 may be configured so that TTM fluid 112 may flow through the porous wall 847, i.e., through the pores 848 of the porous wall 847. Consequently, TTM fluid 112 may flow through the porous wall 847 from the inner tube flow area 845 to the annular flow area 836 and from the annular flow area 836 into the inner tube flow area 845.

In use, the longitudinal velocity of the TTM fluid 112 may change along the length of the filter 800. As the volumetric TTM fluid 112 flow through the filter is constant, the longitudinal velocity of the TTM fluid 112 may be at least partially defined by the flow areas of the filter 800 as described below. The TTM fluid 112 may enter the filter 800 at a first longitudinal velocity 851 and decrease along the diffuser so that the TTM fluid 112 enters the inner tube at a second velocity 852 less than the first longitudinal velocity 851. At this point, a portion of the TTM fluid 112 may flow through the porous wall 847 from the inner tube flow area 845 into the annular flow area 836 to divide the fluid flow into a third velocity 853 within the inner tube flow area 845 and a fourth velocity 854 within the annular flow area 836. The fourth velocity 854 may be less than the third velocity 853. A portion of the TTM fluid 112 may then flow back into the inner tube flow area 845 from the annular flow area 836 to define a fifth velocity 855 along the inner tube flow area 845 which may be about equal to the second velocity 852. The TTM fluid 112 may then proceed along the nozzle 820 to define a sixth velocity 856 exiting the filter 800. In some embodiments, the first velocity 851 and the sixth velocity 856 may be about equal.

The filter 800 may be configured to remove harmful bacteria and viruses from the TTM fluid 112 using sedimentation principles. In use, the filter 800 may be oriented horizontally so that the direction of fluid flow through the filter 800 is perpendicular to a gravitational force 865. In some instances, bacteria, viruses, and other particles within the TTM fluid 112 may have a greater density than the TTM fluid 112 and as such may be urged by the gravitational force 865 (i.e., sink) in a direction perpendicular to the fluid flow direction. In some instances, particles within the inner tube flow area 845 may sink toward and through the porous wall 847 into the annular flow area 836. Particles within the annular flow area 836 may then sink toward an inside surface 831 of the body 830 and become trapped adjacent the inside surface 831. The geometry of the filter 800 may be configured to allow 0.2-micron bacteria/virus particles to fall out of the flow of TTM fluid 112 and become trapped along the inside surface 831.

In some embodiments, the filter 800 may be configured so that flow of TTM fluid 112 from the inner tube flow area 845 into the annual flow area 836 my drag particles through the porous wall 847. In some embodiments, the inlet flow area 811, the inner tube flow area 845, and the annual flow area 836 may be sized so that the third velocity 853 is less than about 50 percent, 25 percent, or 10 percent of the first velocity 851 or less. In some embodiments, the body 830 and the inner tube 840 may be configured so that the fourth velocity 854 is less than the third velocity 853. In some embodiments, the fourth velocity 854 may less than about 50 percent, 25 percent, or 10 percent of the third velocity 853 or less.

In some embodiments, the filter 800 may be configured so that the flow within the inner tube flow area 845 is laminar flow, i.e., so that the velocity of the fluid flow adjacent to or in close proximity to an inside surface 841 of the porous wall 847 is less than the velocity at a location spaced away from the inside surface 841. In such an embodiment, the particles may more readily sink toward and through the porous wall 847.

In some embodiments, the filter 800 may be configured so that the fluid flow within the annual flow area 836 is laminar flow, i.e., so that the velocity of the fluid flow adjacent to or in close proximity to inside surface 831 of the body 830 is less than the velocity at a location spaced away from the inside surface 831. In such an embodiment, the particles may more readily sink toward and be trapped along the inside surface 831.

The filter 800 may include three components including the inner tube 840 an inner body shell 838, and an outer body shell 839. Each of the three components may be formed via the plastic injection molding process. Assembly of the filter 800 may include capturing the inner tube 840 within the inner body shell 838 and the outer body shell 839 and sliding the inner body shell 838 into the outer body shell 839 wherein the fit between the inner body shell 838 and the outer body shell 839 is an interference fit.

In some embodiments, the filter 800 may be disposed within a thermal pad such as the pad 121. FIG. 8C shows a detail cross-sectional view of the pad 121 including the filter 800 disposed within the fluid containing layer 420. The filter 800 is coupled in line with the internal fluid conduit 426 within the fluid containing layer 420 so that TTM fluid 112 circulating within the pad 121 passes through the filter 800. The filter 800 may be sized so that the inlet flow area 811 and the outlet flow area 821 are similar to a cross-sectional flow area of the internal flow path 426 within the fluid containing layer 420.

In some embodiments, a thickness of the fluid containing layer 420 may increase adjacent the filter 800 to accommodate a body diameter 864 of the filter 800. To further accommodate the body diameter 864, the insulation layer 410 and/or the thermal conduction layer 430 may include internal depressions 862, 863, respectively.

In some embodiments, one or more filters 800 may be disposed in line with the flow of TTM fluid 112 at other locations of the TTM system 100. In some embodiments, one or more filters 800 may be disposed within the TTM module 110. In some embodiments, one or more filters 800 may be disposed in line with the fluid conduits (e.g., the fluid delivery conduit 121A or the fluid return conduit 212B).

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A targeted temperature management (TTM) system, comprising:
a TTM module configured to provide a TTM fluid;
a thermal pad configured to facilitate thermal energy transfer between the TTM fluid and a patient, the pad comprising:
a pad portion configured for placement on the patient
a fluid delivery conduit extending away from the pad portion, the fluid delivery conduit including a delivery conduit connector at a proximal end thereof; and
a fluid return conduit extending away from the pad portion, the fluid return conduit including a return conduit connector at a proximal end thereof; and
a fluid delivery line (FDL) including a fluid delivery lumen and a fluid return lumen, the lumens extending from a proximal end to a distal end of the FDL,
wherein the FDL is coupled with the TTM module at the proximal end and includes an FDL hub at the distal end, the hub comprising:
a delivery hub connector coupled with the delivery conduit connector;
a return hub connector coupled with the return conduit connector; and
a locking mechanism selectively configurable between a release configuration and a lock configuration,
wherein when the locking mechanism is in the lock configuration, the locking mechanism prevents at least one of separation of the delivery conduit connector from the delivery hub connector or separation of the return conduit connector from the return hub connector.

2. The system according to claim 1, wherein when the locking mechanism is in the lock configuration, the locking mechanism prevents separation of the delivery conduit connector from the delivery hub connector and separation the return conduit connector from the return hub connector.

3. The system according to claim 1, wherein the locking mechanism comprises a rotatable knob, and wherein transitioning the locking mechanism from the release configuration to the lock configuration comprises rotating the knob from a first angular position to a second angular position.

4. The system according to claim 1, wherein the delivery conduit connector is attached to the return conduit connector.

5. The system according to claim 1, wherein the delivery hub connector and/or the return hub connector comprises a valve configured to selectively allow and prevent flow of fluid through the delivery hub connector or the return hub connector, respectively.

6. The system according to claim 5, wherein connecting the delivery conduit connector to the delivery hub connector opens the valve of the delivery hub connector.

7. The system according to claim 5, wherein disconnecting the delivery conduit connector from the delivery hub connector closes the valve of the delivery hub connector.

8. The system according to claim 5, wherein the valve of the delivery hub connector is closed unless the delivery conduit connector is connected to the delivery hub connector.

9. The system according to claim 5, wherein the valve comprises a septum extending across a lumen of the delivery hub connector.

10. The system according to claim 9, wherein the septum comprises a slit configured to be disposed between an open configuration and a closed configuration, and wherein:
when the slit is in the open configuration, flow of TTM fluid through the delivery hub connector is allowed, and
when the slit is in the closed configuration, flow of TTM fluid through the delivery hub connector is prevented.

11. The system according to claim 1, wherein:
the thermal pad comprises a radio frequency identification (RFID) tag configured to provide pad identification data,
the TTM module comprises an RFID sensor configured to receive pad identification data from the RFID tag, and
pad identification logic stored in memory of the TTM module is configured to alert the clinician according to an identification of the pad.

12. The system according to claim 1, wherein the thermal pad comprises a filter in fluid communication with the fluid delivery conduit such that TTM fluid passing through the fluid delivery conduit passes through the filter.

13. The system according to claim 12, wherein the filter comprises a porous wall oriented parallel to a continuous flow path through the filter.

14. A medical pad for exchanging thermal energy between a targeted temperature management (TTM) fluid and a patient, the pad comprising:
a pad portion configured for placement on the patient;
a fluid delivery conduit extending away from the pad portion, the fluid delivery conduit including a delivery conduit connector at a proximal end thereof; and
a fluid return conduit extending away from the pad portion, the fluid return conduit including a return conduit connector at a proximal end thereof; and
an RFID tag configured to provide pad identification data to an RFID sensor.

15. The pad according to claim 14, wherein the RDIF tag is attached to the pad portion.

16. The pad according to claim 14, further comprising:
a fluid containing layer configured to contain circulating TTM fluid therein; and
an insulation layer coupled with the fluid containing layer,
wherein the RFID tag is disposed between the insulation layer and the fluid containing layer.

17. The pad according to claim 14, wherein the delivery conduit connector and the return conduit connector are attached together.

18. The pad according to claim 14, wherein the delivery conduit connector and the return conduit connector are configured to couple with a fluid delivery line of a TTM module to establish fluid communication of the fluid delivery conduit and the fluid return conduit with the fluid delivery line.

19. The pad according to claim 14, wherein at least one of the delivery conduit connector or the return conduit connector are configured to be locked to the fluid delivery line to prevent separation of the at least one of the delivery conduit connector or the return conduit connector from the fluid delivery line.

20. The pad according to claim 14, further comprising a filter in fluid communication with the fluid delivery conduit such that TTM fluid passing through the fluid delivery conduit passes through the filter.

21. The pad according to claim 20, wherein the filter comprises a porous wall oriented parallel to a continuous flow path through the filter.

22-31. (canceled)
